(19) United States
(12) Patent Application Publication
(10) Pub. No.: US 2009/0054798 A1
(43) Pub. Date: Feb. 26, 2009

(54) RESPIRATION MONITOR AND METHOD OF ITS OPERATION

(75) Inventors: Mark Varney, Hampshire (GB); Michael Ernest Garrett, Surrey (GB); Deryk Williams, Surrey (GB)

Correspondence Address: Kimberly A. Chasteen, PO Box 1243, Yorktown, VA, 23692, US

(73) Assignee: ASTHMA ALERT LIMITED, Surrey (GB)

(21) Appl. No.: 11/791,387

(22) PCT Filed: Nov. 22, 2005
(86) PCT No.: PCT/GB2005/004495

§ 371 Date: Dec. 6, 2007

(30) Foreign Application Priority Data
Nov. 22, 2004 (GB) 0425670.7

(51) Int. Cl. A61B 5/087; A61B 5/08

(52) U.S. Cl. 600/532; 600/538

(57) ABSTRACT

An apparatus for monitoring the respiration of a subject, the apparatus comprises means for detecting the concentration of each of oxygen, carbon dioxide and nitric oxide in an inspired and/or exhaled gaseous stream of a subject; and a display for displaying the concentration of each of oxygen, carbon dioxide and nitric oxide. A method for monitoring the respiration of a subject comprises analysing the composition of the inspired and/or exhaled gas of the subject and determining the concentration of carbon dioxide, oxygen and nitric oxide in the gas; and displaying the results of the analysis on a display to provide an indication of the respiratory state of the subject. A method of determining the extent of resuscitation of a patient comprises measuring the concentration of carbon dioxide, oxygen and nitric oxide in the inspired gas stream and/or exhaled gas stream of the subject.

The present invention relates generally to the field of monitoring the respiration of a patient, in particular to the field of resuscitation. Specifically, the present invention relates to a method and apparatus for monitoring the respiration of a patient, in particular to determine whether the patient is alive.

Respiratory monitors are known in the art and a number of systems are known for monitoring the composition of respiratory gases. U.S. Pat. No. 5,003,985 and WO 02085207 describe an algorithmic approach to the analysis of respiratory gases that follow the end-tidal concentrations of carbon dioxide and oxygen through both the inhalation and exhalation cycles. The resultant waveforms are appropriately interpreted and specifically used to control anaesthesia of the patient.

US 2004,215,112 discloses a similar method for monitoring the end-tidal concentration of carbon dioxide of a patient, which is used to mechanically control chest compression during CPR, to control drug delivery, or control a defibrillator.

US 2003,029,453 describes a complete emergency life support system including a patient ventilator for mechanical breathing assistance. Inhalation and exhalation ratios of carbon dioxide and pulse oximetry measurements are taken as the main measures of ventilation effectiveness.

DE 19953866 describes a method of calculating the respiration rate (that is work) from the amount of oxygen consumed during exercise.

While respiratory monitors are known, the prior art devices only sample a limited aspect of a subject's respiration, or display a limited number of measured parameters. Further, the current respiratory monitors are not used for resuscitation purposes. In addition, the known respiratory monitors do not allow a user to determine the efficiency or extent of artificial ventilation. In particular, no respiratory monitor has been used to sense nitric oxide as an indicator of vital signs.

Following and monitoring the progress of resuscitation of a patient or subject is difficult. While a subject may be artificially ventilated, there is no adequate method to determine respiration or other important physiological functions without relying upon human intervention. Accordingly, there is a need for a device to monitor the respiration of a subject or patient in order to determine the extent of resuscitation.

Accordingly, in a first aspect, the present invention provides an apparatus for monitoring the respiration of a subject, the apparatus comprising:

means for detecting the concentration of each of oxygen, carbon dioxide and nitric oxide in an inspired and/or exhaled gaseous stream of a subject; and
a display for displaying the concentration of each of oxygen, carbon dioxide and nitric oxide.

The key to the present invention is that in a healthy patient or subject, there should be present elevated concentrations of carbon dioxide and nitric oxide in the exhaled breath, while oxygen concentrations should be reduced, compared with the inhaled gas stream. In other words, oxygen should be consumed from the inspired breath.

The present invention provides an electrochemical apparatus for use in determining tidal concentrations of respiratory gases, especially end-tidal concentrations, in particular carbon dioxide, oxygen and nitric oxide, in order to determine the presence or absence of life in the subject and to generally assisting monitoring respiration and general physiological condition. In this respect, the term ‘end-tidal’ is a reference to the composition of the gas stream at the end of the exhaled breath of the subject or patient. During normal breathing of a healthy subject, the gas exhaled at the end of the breath will contain the maximum concentration of carbon dioxide and nitric oxide and the lowest concentration of oxygen.

All three gases, carbon dioxide, nitric acid and oxygen may be monitored by electrochemical sensors specifically adapted to each gas.

Electrochemical oxygen (O₂) sensors are well known in the field. They are commonly constructed with a gold cathode, electrically polarised relative to a silver anode. A high ionic conductivity alkaline electrolyte facilitates the direct reduction of oxygen molecules. A sensor for nitric oxide (NO) can be based on either the direct oxidation or reduction of the gas to nitrous oxide or ammonia, respectively, at a carbon electrode. NO may also be electrochemically measured indirectly by its reaction with an intermediate molecule, such as a tetracyclic polypyrrole or porphyrin.

Carbon dioxide is difficult to reduce electrochemically. A suitable electrochemical sensor for the detection of carbon dioxide (CO₂) may instead be based on the Severinghaus principle, whereby the dissolution of carbon dioxide gas changes the pH through the formation of carbonic acid. The change in pH may be directly measured by a pH electrode immersed within the electrolyte. However, it is important that the electrolyte is neutral and not pH buffered, as a buffer would otherwise resist the change in pH caused by the ingress of the CO₂.

The apparatus preferably measures real-time inspired and tidal exhalation concentrations of respiratory gases, in particular carbon dioxide, oxygen and nitric oxide. In this respect, a reference to ‘real-time’ is to mean that the apparatus will display the concentrations of the gases being detected with minimal or substantially no delay, thus giving an indication of the composition of the gas stream passing through the device at that time.

In one embodiment, the apparatus is adapted to measure the concentration of each of oxygen, carbon dioxide and nitric oxide in a breath-on-breath regime. In this respect, ‘breath-on-breath’ refers to the concentrations of the individual gas molecules towards the end of an inhalation-exhalation cycle. During the initial phase of an exhalation, the carbon dioxide concentration in the breath is negligible. The first exhaled gases generally carry air from so-called “dead space” within the subject's body, such as the trachea and bronchi in which no gas exchange takes place. During the later phase of exhalation, and as gases from the alveoli are expelled with air from the dead space, the concentration of carbon dioxide in the breath rises. When the dead space gases are mostly expelled, the concentration of carbon dioxide begins to reach a plateau (the “end-tidal” phase). The end-tidal concentration is a more accurate reflection of the pulmonary concentration, as this is least affected by mixing with inspiratory gases. The measurement of a sequence (or series) of end-tidal concentrations will thereby reveal any long-term change or trend in respiration of a subject breathing through the apparatus.

The display may be configured to show a range of different data relating to the composition and other properties of the gas stream being monitored. In one embodiment, the display is adapted to display the concentrations of the said gases for both the inspired gas stream and exhaled gas stream. In particular, the apparatus is adapted to measure the concentration of the said gases in consecutive inspired and exhaled breaths of the subject, the display being adapted to show a comparison of the composition of the inspired and exhaled streams. A comparison of the inspired and exhaled gas concentrations provides relative estimates of the gas exchange from the pulmonary (arterial) circulation across the lung surface of the subject or patient. In particular, this may be calculated as being a ratio of the compositions of the gases in the inspired and exhaled streams.

Comparison of the inspired/expired concentrations of carbon dioxide, oxygen and nitric oxide with those of population normal values provides a method of determining the presence of life in the subject or patient, while minimising the danger of a misdiagnosis, such as a power failure resulting in a loss of signal, which may be interpreted as being the absence of life in the subject. Importantly, the opposing change in signals indicates the correct functioning of the sensors, and qualifies the presence of the vital signs. The apparatus in this embodiment compares a number of parameters characteristic of the subject's breath with a stored library of those parameters expected from normal signals and waveforms stored in a memory. Analysis of the of the outputs and/or waveforms of the apparatus as simultaneously measured by the carbon dioxide, oxygen and nitric oxide sensors provides the operator with diagnostic information about the extent of resuscitation of the subject or patient.

For carrying out the analysis of the outputs received from the gas sensors, the apparatus preferably comprises a processor or microcontroller. This may perform such functions as generating the images representing the data on the display, calculating the ratios of gas concentrations between inspired and exhaled streams, comparing the output from the sensors with data stored in the library relating to representative values of the normal population, and the like.

In one embodiment, the apparatus further comprises a means for measuring the differential pressure of the gas flow, and a processor adapted to calculate the volume flow rate of gas passing through the apparatus. This measurement may be used in an estimation of the volume of inspired and/or exhaled gas per unit time throughout the breathing cycle. This allows for calculation of the inspiratory/expiratory volumes, and thus the mass of gases per unit time, per breath, per multi-breath cycle and over time. These data may used in an algorithmic approach to determine the condition of the subject or patient. In particular, these data can be used to determine if each breath is a spontaneous (that is a natural) breath, or a breath induced artificially by a ventilator. This indication can be important when a determination is to be made as to the reliance a patient or subject has on artificial respiration means, such as a ventilator or the like.

The apparatus may also include sensors to determine the temperature and humidity of the gas stream. Variations in the temperature and humidity between the inspired and exhaled gas streams may then be determined. It should be noted that temperature and humidity are the two primary variables of gas density. Alternatively, the inhaled gas may be adjusted in temperature and humidity to match those of the exhaled breath.

In a further aspect, the present invention provides a method of monitoring the respiration of a subject, the method comprising analysing the composition of the inspired and/or exhaled gas of the subject and determining the concentration of carbon dioxide, oxygen and nitric oxide in the gas; and displaying the results of the analysis on a display to provide an indication of the respiratory state of the subject.

The concentration of each of oxygen, carbon dioxide and nitric oxide is preferably measured in real-time. One preferred regime is one in which the concentration of each of oxygen, carbon dioxide and nitric oxide in measured in a breath-on-breath regime.

The method preferably comprises measuring the tidal concentration of oxygen, carbon dioxide and nitric oxide. In a preferred embodiment, the end-tidal concentration of these gases is measured for the subject.

The method may be employed to determine and display the concentrations of the aforementioned gases in either the inspired gas stream or the exhaled gas stream. A preferred method is one in which the concentrations of the said gases for both the inspired gas stream and exhaled gas stream are determined. The concentration of the said gases in consecutive inspired and exhaled breaths of the subject may be measured, with the display preferably showing a comparison of the composition of the inspired and exhaled streams.

As noted hereinbefore, a particularly useful indicator of the respiratory state of the subject or patient is the ratio of concentrations of the gases in the inspired and exhaled gas streams. Accordingly, the method preferably further comprises calculating the ratio of each of the said gases in the inspired gas stream and the exhaled gas stream. These ratios may be compared with stored data relating to population normal values of the ratios, from which an indication of the extent of resuscitation of the subject or patient may be obtained. In such a case, the method preferably includes providing an indication of the extent of resuscitation on the display.

The method may also comprise measuring the differential pressure of the gas flow. In addition, other properties of the gas stream may be determined, such as the temperature and/or humidity of the gas stream. In one preferred embodiment, the method includes adjusting the temperature and/or humidity of the gas stream, in particular that of the inspired gas to match that of the exhaled gas stream being measured.

Any suitable sampling and sensing regime may be employed, provided that adequate data regarding the condition of the subject are provided to the operator. In a preferred method the concentrations of the said gases are measured continuously or periodically. If measured periodically, the concentrations of the said gases are measured periodically every 1 to 100 milliseconds, preferably from 5 to 50 milliseconds, most preferably about every 10 milliseconds.

In a further aspect, the present invention comprises a method of determining the resuscitation state of a subject, the method comprising measuring the concentration of carbon dioxide, oxygen and nitric oxide in the inspired and/or the exhaled breath of the subject.

Preferably, the resuscitation state is determined from measuring the concentration of the said gases in both the inspired gas stream and exhaled gas stream of the subject. In one preferred embodiment, the method further comprises calculating a ratio of the concentrations of the said gases in the inspired and exhaled gas streams, comparing the ratios thus determined with population normal values for the ratios, and determining the extent of resuscitation of the subject from the results of the comparison.

The method of determining the extent of resuscitation of a subject may comprise the features hereinbefore described.

Embodiments of the present invention in its various aspects will now be described having reference to the accompanying figures, in which:

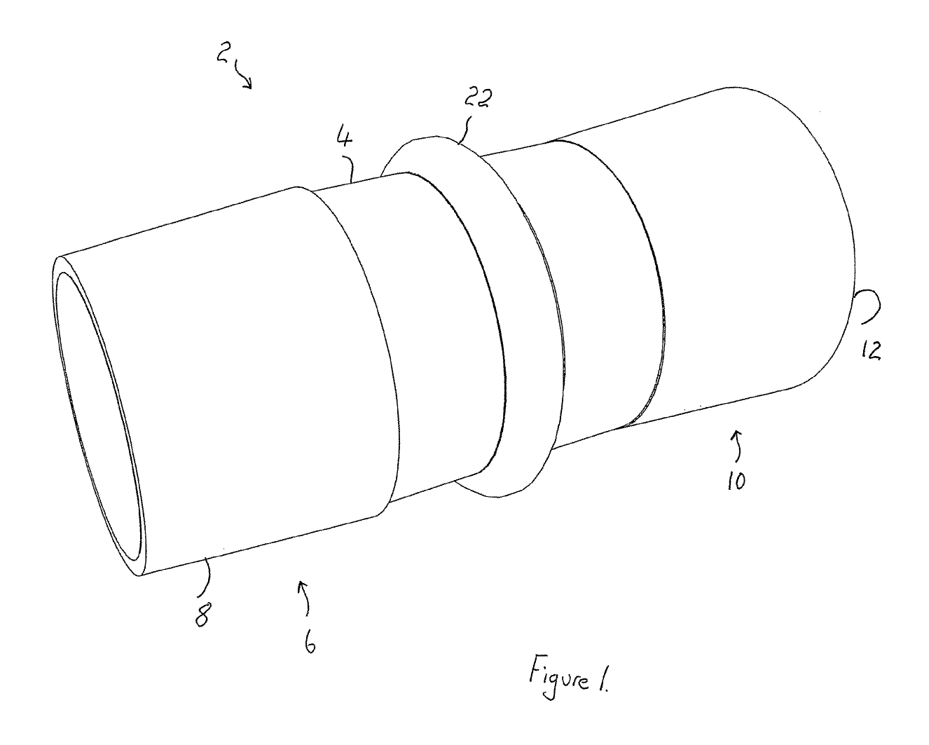

FIG. 1 is a perspective view of a sensor body for measuring a respiratory gas;

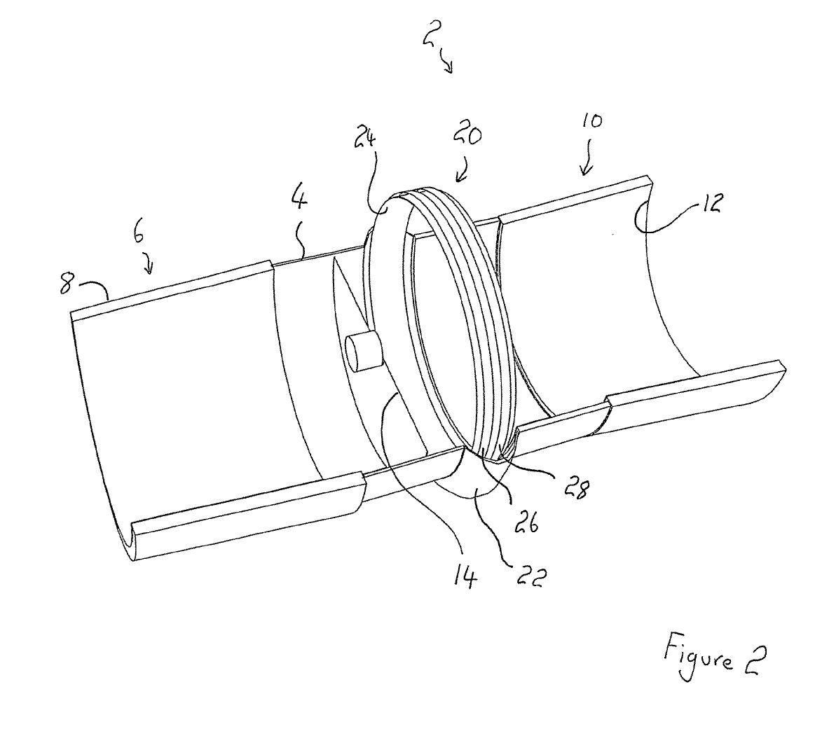

FIG. 2 is a cut-away view of the sensor of FIG. 1;

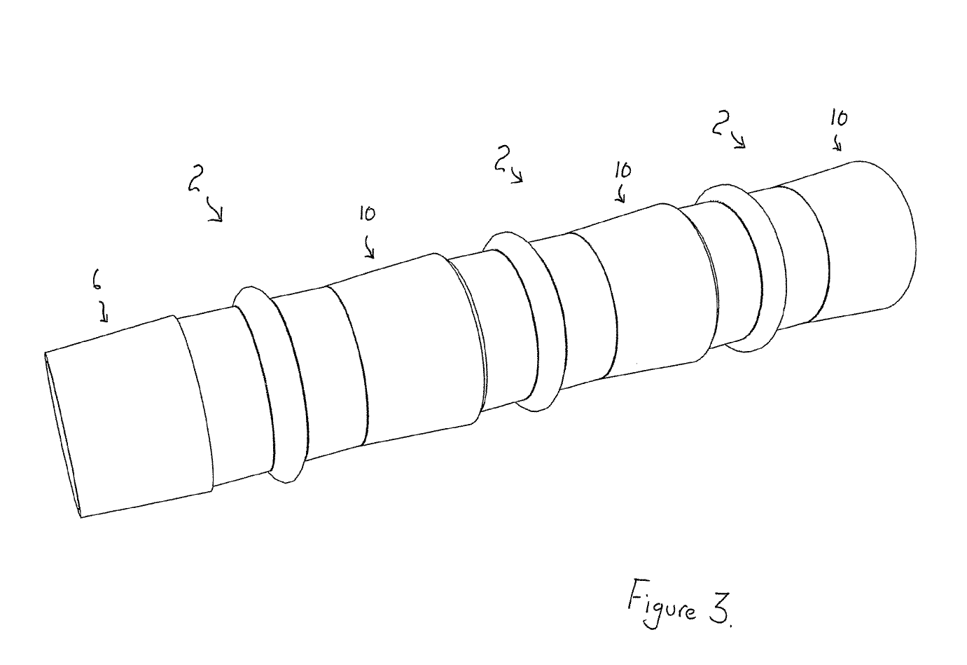

FIG. 3 is a perspective view of an assembly of a plurality of sensor bodies of FIG. 1; and

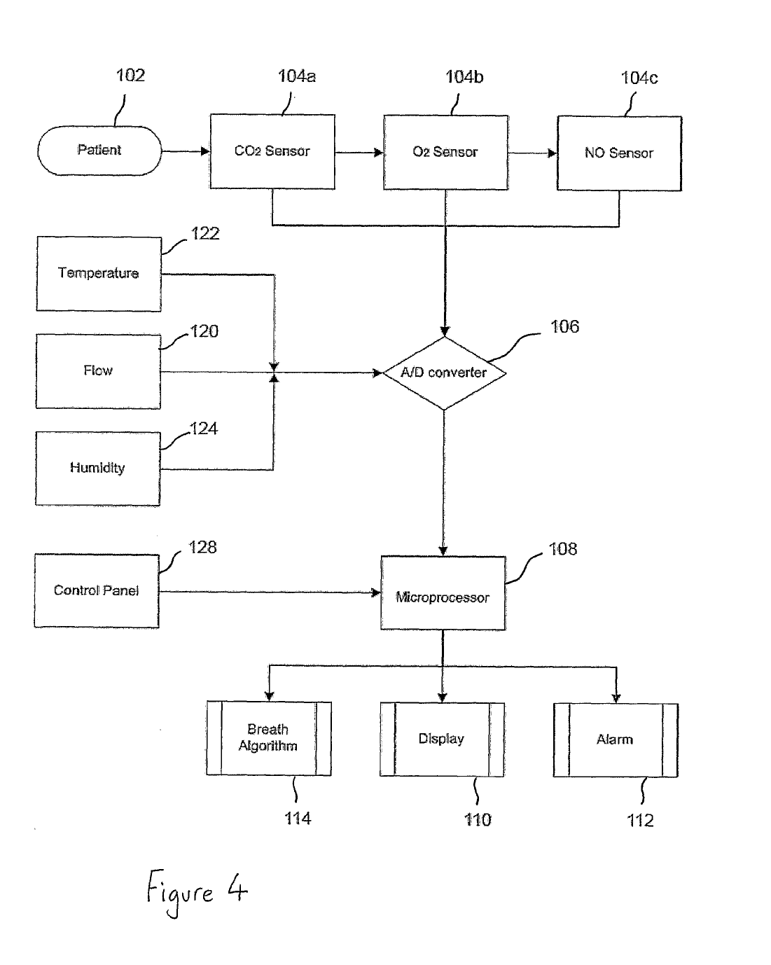

FIG. 4 is a flow diagram that illustrates an overview of the interconnection of the sensor bodies and their connection to a microcontroller.

Referring to FIG. 1, there is shown a perspective view of an apparatus, generally indicated as 2, for monitoring the respiration of a patient or subject. The apparatus comprises a generally cylindrical, tubular adaptor or housing 4, forming a conduit through which a gas stream may be caused to pass so as to be analysed. The housing 4 has a first end portion 6 having an outer diameter and profile 8 and a second end portion 10 having an inner diameter and profile 12. The outer diameter and profile 8 and the inner diameter and profile 12 are sized such that the first end portion 6 of one housing may be inserted into the second end portion 10 of a second housing in a push fit. A common terminology is to refer to the first end portion 6 as a male end and the second end portion 10 as a female end. In this way, a series of two or more apparatus 2 may be connected together in line, allowing a series of the apparatus to be used simultaneously. Such an assembly of three apparatus is shown in FIG. 3. Each of the apparatus 2 in the assembly in FIG. 3 comprises a sensor for detecting a different one of the three respiratory gases oxygen, carbon dioxide and nitric oxide.

Reference is now made to FIG. 2, showing a cut-away view of the apparatus of FIG. 1. The housing 4 is provided with a one-way valve 14 of conventional construction. The presence of the one-way valve 14 allows the apparatus to measure the gas concentration on either the inhalation or the exhalation phases of the patient or subject. If the one-way valve 14 is removed, the apparatus may be used to measure gas concentrations for both inhalation and exhalation phases, in order to follow the changes in gas concentration throughout the pattern of breathing. Accordingly, the one-way valve 14 is preferably made to be easily removed from the housing 4.

The apparatus 2 further comprises a sensor, generally indicated as 20, housed in a central portion 22 of the housing of larger diameter. It is important that the sensor 20 does not block or occlude in any way the conduit through the housing 4, in order not to alter the flow of gas and disturb the measurements. Accordingly, the sensor 20 is arranged as a ring around the central conduit of the housing 4. A membrane 24 is arranged to be flush with the inner surface of the housing 4.

In a similar respect, it is important to minimise any phase lag and dead volumes with the housing 4, which could otherwise lead to mixing and dilution of the gases in the exhalation stream.

The sensor 20 is selective towards one of the respiratory gases carbon dioxide, oxygen and nitric oxide. With a single sensor 20 retained inside the housing 4, at least three such assemblies will be required to be linked end to end, as described hereinbefore, in order to provide the apparatus with the capacity to sense all three of the aforementioned respiratory gases. Alternatively, the housing 4 may be provided with a plurality of separate central portions 22, (for example, two, three or more) each housing a sensor 20 for detecting the concentration of one of the said respiratory gases. The apparatus 2 would comprise a combination of the requisite number and type of sensors 20 within their housing. One preferred alternative apparatus comprises three separate central portions 22.

The sensor 20 is formed as an electrochemical cell. The electrochemical cell comprises a counter electrode 26 and one working electrode 28 housed within the central portion 22. Each of the electrodes 26 and 28 is formed as a ring to extend around the conduit in the housing 4 within the central portion 22. A suitable electrolyte is provided in the central portion 22 to provide an electrochemical link or bridge between the two electrodes 26 and 28. A semi-permeable membrane extends between the two electrodes 26 and 28.

The electrodes 26 and 28 may comprise any suitable metal or alloy of metals, with the proviso that the electrode does not react with the electrolyte or any of the substances present in the gas stream. Preference is given to metals in Group VIII of the Periodic Table of the Elements (as provided in the Handbook of Chemistry and Physics, 62^{nd} edition, 1981 to 1982, Chemical Rubber Company). Other suitable metals include copper, silver and gold. Preferably, each electrode is prepared from gold or platinum.

The electrolyte may be any suitable composition and is preferably in the form of a liquid or a gel. Suitable electrolyte compositions typically include water and a salt such as a salt of a Group I metal, in particular, sodium or potassium. Suitable salts are the halides, in particular chlorides. The salts act to aid the ionic conductance (signal) through the electrolyte. Gel-type electrolytes would also have a low concentration of cross-linked polymer, such as polyacrylamide or polysaccharide.

An alkaline electrolyte facilitates the direct reduction of oxygen to hydroxide, through the direct electrochemical reduction of the gas, according to the following equation:

O₂+2H₂O+4e⁻=4OH⁻

A sensor for nitric oxide (NO) can be based on either the direct oxidation or reduction of the gas to nitrous oxide or ammonia, respectively, at a carbon (working) electrode. However, this may result in a sensor that is less selective towards the gas than desirable, as the reaction potential necessary for oxidation or reduction of the nitric oxide could also include the electroreaction of other (interfering) molecules. NO may therefore be electrochemically measured indirectly by its reaction with an intermediate molecule, such as a metal (tetracyclic) polypyrrole or porphyrin. These “organometallic complexes” may be simply formulated into suitable electrolyte compositions. However, as the biologic concentrations of NO are extremely low, the overall design of an NO sensor should maximise the signal and reduce any potential noise. The design of electrode which have these organometallic complexes deposited as layers on their surface (a “modified” electrode) is therefore attractive and provides one ideal strategy. Substantial improvements in signal-to-noise will further result from the use of microelectrodes in the design.

A suitable electrochemical sensor for the detection of carbon dioxide (CO₂) may be based on the Severinghaus principle, whereby the dissolution of carbon dioxide gas changes the pH of a high ionic conductivity electrolyte. The change in pH may be directly measured by a pH electrode immersed within the electrolyte. However, it is important that the electrolyte is neutral and not pH buffered, as a buffer would otherwise resist the change in pH caused by the ingress of the CO₂.

In all the above cases, a thin polymeric membrane would be stretched across the face of the electrodes to retain the electrolyte, and minimise the loss of water by evaporation. By the appropriate selection of the membrane material, the permeation characteristics (permeability coefficient) of the membrane will afford an additional degree of selectivity towards an individual gas molecule.

In addition, the apparatus 2 comprises a potentiostat (not shown for clarity) to apply a voltage to the counter electrode and measure the current at the working electrode.

As an alternative to having the sensor 20 in contact with the gas stream passing through the central passage of the housing 4, the housing may be provided with an opening to provide a side stream of gas, which may the subject of the analysis.

In operation, gas from the gas stream passing through the housing 4 passes through the membrane 24 and diffuses into the electrolyte. The current measured at the working electrode 28 as a result of the voltage applied to the counter electrode will depend upon the concentration of the target component in the electrolyte, which in turn is dependent upon the concentration of the target component in the gas stream passing through the housing. Accordingly, the current measured at the working electrode 28 may be correlated to provide an indication of the concentration of the target component in the gas stream being analysed.

The control, acquisition and processing of the signals received from the sensor 20 is carried out be a processor or microcontroller. The microcontroller is provided with suitable peripheral analog-to-digital and digital-to-analog converted devices, in order to process the analog current signals received from the working electrode and provide suitable signals to a display device. The microcontroller is capable of converting the signals generated by the sensor into meaningful indications of units of concentration for use by the operator. The microcontroller is also provide with a suitable memory for storing data, in order to provide a historical indication of the respiratory condition of the patient or subject. In addition, the microcontroller is provided with a library of stored reference values of concentration or ratios representative of a normal population, which are retrieved and processed with the signals received from the sensor when an indication of the extent of resuscitation of the patient or subject is required.

One embodiment of the control and display system of the present invention is shown, schematically, in FIG. 4. The patient 102 breaths into an apparatus according to the present invention, for example the apparatus shown in FIG. 1 and described hereinbefore. As shown in FIG. 4, the apparatus comprises three sensors, 104a, 104b and 104c, capable of detecting the presence of carbon dioxide, oxygen and nitric oxide respectively. The signals output by the sensors 104a, 104b and 104c are conveyed to an analog/digital converter 106. The digital signals thus produced are fed to a microprocessor 108 for processing. The microprocessor 108 may be programmed to carry out one or more of the various functions hereinbefore described.

The microprocessor 108 can output a range of signals and generate a range of displays, depending upon the information required to be conveyed to the operator. As shown in FIG. 4, the microprocessor 108 will output signals to control a display 110, operate an alarm 112 when required, and control a breath algorithm 114.

The breath algorithm compares the individual gas concentrations to the expected values in the exhaled breath of the normal population. The microprocessor collects continuous data from the individual sensors and convert these data into concentration values. The readings are continuously assessed for any change from the normal expected values, and will be flagged if any particular reading departs from that value. Statistical or regression analysis may be used to quantify the change or trend in the signal. Of particular interest is that during CPR the signals will change in a consistent direction according to the condition of the patient. If, for example, the subject enters into respiratory distress, the carbon dioxide concentration will drop from its normal value of approximately 5%, the oxygen levels will rise back to normal atmospheric levels of approximately 20%, and the nitric oxide concentrations will fall to 0%. The systematic change in signal will also distinguish important changes due to respiratory reasons from instrumental changes, such as battery failure, when all the signals fall to 0.

In addition, in the arrangement shown in FIG. 4, the system is provided with a detector for measuring each of the gas flowrate, the temperature and humidity of the gas stream, identified as 120, 122 and 124 respectively. The output signals from these detectors 120, 122, 124 are also fed to the microprocessor, via a suitable analog/digital converter, if required (but not shown for clarity). These signals may be included in the processing operations performed by the microprocessor 108.

Finally, the system shown in FIG. 2 is provided with a control panel 128, by which the operator may control the operation of the microprocessor, for example to select the mode or options of the display, reset the alarm, store or retrieve data, or the like. The control panel 128 may be of any suitable form, for example a simple keypad, the construction of which is known in the art. Alternatively, the control panel 128 may be incorporated in the display 110 in the form of a touch-screen system, again known in the art.

The methods of digital sampling and digitization of analog signals received from the sensor that are suitable and may be employed are known in the art.

In addition, the apparatus may be provided with a means for measuring the flow rate of the gas stream through the housing 4 and the temperature and humidity of the gas stream in the housing. Again, suitable means for making such measurements are known in the art.

It will be appreciated that the aforementioned apparatus and its use of an electrochemical cell to measure the concentration of carbon dioxide, oxygen and nitric oxide is a preferred manner of carrying out the method of the present invention. However, alternative technologies that may be used to measure the gas concentrations include (but are not necessarily restricted to) infrared spectroscopy, mass spectroscopy and chemiluminescence.

1. An apparatus for monitoring the respiration of a subject, the apparatus comprising:
means for detecting the concentration of each of oxygen, carbon dioxide and nitric oxide in an inspired and/or exhaled gaseous stream of a subject; and
a display for displaying the concentration of each of oxygen, carbon dioxide and nitric oxide.

2. The apparatus according to claim 1, wherein the apparatus is adapted to measure the concentration of each of oxygen, carbon dioxide and nitric oxide in real-time.

3. The apparatus according to claim 2, wherein the apparatus is adapted to measure the concentration of each of oxygen, carbon dioxide and nitric oxide in a breath-on-breath regime.

4. The apparatus according to any preceding claim, wherein the apparatus is adapted to measure the tidal concentration of oxygen, carbon dioxide and nitric oxide.

5. The apparatus according to any preceding claim, wherein the display is adapted to display the concentrations of the said gases for both the inspired gas stream and exhaled gas stream.

6. The apparatus according to claim 5, wherein the apparatus is adapted to measure the concentration of the said gases in consecutive inspired and exhaled breaths of the subject, the display being adapted to show a comparison of the composition of the inspired and exhaled streams.

7. The apparatus according to claim 6, further comprising a processor to calculate the ratio of each of the said gases in the inspired gas stream and the exhaled gas stream.

8. The apparatus according to claim 7, further comprising a memory adapted to store data relating to population normal values of the said ratios, the processor further providing a comparison between the measured concentrations and the data stored in the memory.

9. The apparatus according to claim 8, wherein the processor is adapted to provide an indication of the extent of resuscitation of the subject.

10. The apparatus according to any preceding claim, further comprising means for measuring the differential pressure of the gas flow, and a processor adapted to calculate the volume flow rate of gas.

11. The apparatus according to any preceding claim, further comprising means for measuring the temperature and/or humidity of the gas stream.

12. The apparatus according to any preceding claim, further comprising means for adjusting the temperature and/or humidity of the gas stream.

13. The apparatus according to any preceding claim, further comprising a plurality of sensor modules, each sensor module comprising a conduit, a sensor element for detecting the concentration of a component of a gas stream passing through the conduit, the conduits of each module being connected to an adjacent module to provide for analysis of the gas stream by each sensor element.

14. A method of monitoring the respiration of a subject, the method comprising analysing the composition of the inspired and/or exhaled gas of the subject and determining the concentration of carbon dioxide, oxygen and nitric oxide in the gas; and displaying the results of the analysis on a display to provide an indication of the respiratory state of the subject.

15. The method according to claim 14, wherein the concentration of each of oxygen, carbon dioxide and nitric oxide is measured in real-time.

16. The method according to claim 14 or 15, wherein the concentration of each of oxygen, carbon dioxide and nitric oxide in measured in a breath-on-breath regime.

17. The method according to any of claims 14 to 16, wherein the tidal concentration of oxygen, carbon dioxide and nitric oxide is measured.

18. The method according to any of claims 14 to 17, wherein the concentrations of the said gases for both the inspired gas stream and exhaled gas stream are determined.

19. The method according to claim 18, wherein the concentration of the said gases in consecutive inspired and exhaled breaths of the subject is measured, the display showing a comparison of the composition of the inspired and exhaled streams.

20. The method according to claim 18, further comprising calculating the ratio of each of the said gases in the inspired gas stream and the exhaled gas stream.

21. The method according to claim 20, further comprising retrieving from a memory data relating to population normal values of the said ratios, and further providing a comparison between the measured concentrations and the data stored in the memory.

22. The method according to claim 21, further comprising providing an indication of the extent of resuscitation of the subject.

23. The method according to any of claims 14 to 22, further comprising measuring the differential pressure of the gas flow.

24. The method according to any of claims 14 to 23, further comprising measuring the temperature and/or humidity of the gas stream.

25. The method according to claim 24, further comprising adjusting the temperature and/or humidity of the gas stream.

26. The method according to any of claims 14 to 25, wherein the concentrations of the said gases are measured continuously or periodically.

27. The method according to claim 26, wherein the concentrations of the said gases are measured periodically every 1 to 100 milliseconds, most preferably about every 10 milliseconds.

28. A method of determining the resuscitation state of a subject, the method comprising measuring the concentration of carbon dioxide, oxygen and nitric oxide in the inspired gas stream and/or exhaled gas stream of the subject.

29. The method of claim 28, wherein the concentration of the said gases is measured in both the inspired and exhaled gas stream of the subject.

30. The method of claim 29, further comprising calculating a ratio of the concentrations of the said gases in the inspired and exhaled gas streams.

31. The method of claim 30, further comprising comparing the ratios thus determined with population normal values for the ratios, and determining the extent of resuscitation of the subject from the results of the comparison.
